(19) United States
(12) Patent Application Publication
(10) Pub. No.: US 2015/0265221 A1
(43) Pub. Date: Sep. 24, 2015

(54) METHODS AND SYSTEMS FOR DIAGNOSTIC MONITORING

(71) Applicant: Boston Scientific Scimed, Inc., Maple Grove, MN (US)

(72) Inventors: Aiden FLANAGAN, Kilcolgan (IE); Bryan Allen CLARK, Forest Lake, MN (US)

(73) Assignee: Boston Scientific Scimed, Inc.

(21) Appl. No.: 14/641,025

(22) Filed: Mar. 6, 2015

Related U.S. Application Data
(60) Provisional application No. 61/955,431, filed on Mar. 19, 2014.

(51) Int. Cl. A61B 5/00; A61B 5/0205

(52) U.S. Cl. CPC A61B 5/7275; A61B 5/0205; A61B 5/746; A61B 5/6862; A61B 5/686; A61B 5/6852; A61B 5/0809

(57) ABSTRACT

A method of monitoring a lung condition of a patient may include automatically receiving a diagnostic signal from one or more sensors delivered adjacent to or within a lung of the patient. The one or more sensors may be active to conduct diagnostic monitoring of the lung. The method also may include calculating a diagnostic value based on the diagnostic signal, and determining, based on the diagnostic value, a change in the lung condition.

### CROSS-REFERENCE TO RELATED APPLICATION

This patent application claims benefit of priority under 35 U.S.C. §119 to U.S. Provisional Patent Application No. 61/955,431, filed Mar. 19, 2014, the entirety of which is incorporated herein by reference.

### TECHNICAL FIELD

Various embodiments of the present disclosure relate generally to monitoring body conditions and providing alerts if it is determined that medical treatment is necessary. More specifically, the present disclosure relates to methods and systems for diagnostic testing of body conditions, processing the diagnostic test data, and determining and/or predicting the onset of an event requiring medical treatment.

### BACKGROUND

Chronic obstructive pulmonary disease (COPD) includes conditions such as, e.g., chronic bronchitis and emphysema. COPD is estimated to affect about 64 million people worldwide, 15 million of which are in the United States alone, and is currently the third leading cause of death in the United States. The primary cause of COPD is inhalation of cigarette smoke, responsible for over 90% of COPD cases. The economic and social burden of the disease is substantial and is increasing.

Chronic bronchitis is characterized by chronic cough with sputum production. Due to airway inflammation, mucus hypersecretion, airway hyperresponsiveness, and eventual fibrosis of the airway walls, significant airflow and gas exchange limitations result. Emphysema is characterized by the destruction of the lung parenchyma. This destruction of the lung parenchyma leads to a loss of elastic recoil and tethering which maintains airway patency. Because bronchioles are not supported by cartilage like the larger airways, they have little intrinsic support and therefore are susceptible to collapse when destruction of tethering occurs, particularly during exhalation.

Other symptoms of COPD include hyperinflation of portions of the lungs due to air getting trapped in lung tissue and causing the lungs to overinflate. Hyperinflation may also be caused by the air sacs in the lungs losing elasticity and thereby negatively affecting air expulsion from the lungs. In addition, COPD may be associated with formation of voids and bullae, which are large cystic alveolar dilations. The diseased portions may be distributed heterogeneously or homogeneously in the lungs.

Acute exacerbations of COPD (AECOPD) often require emergency care and inpatient hospital care. AECOPDs are defined by a sudden worsening of symptoms (e.g. increase in or onset of cough, wheezing, and sputum changes) that typically last for several days, up to a couple weeks. Bacterial infection, viral infection, or pollutants typically trigger AECOPDs, which manifest quickly into airway inflammation, mucus hypersecretion, and bronchoconstriction, which cause significant airway restriction.

Despite relatively efficacious drugs (e.g. long-acting muscarinic antagonists, long-acting beta agonists, corticosteroids, and antibiotics) that treat COPD symptoms, a particular segment of patients known as “frequent exacerbators” often visit hospital emergency rooms with exacerbations and also have a more rapid decline in lung function, poorer quality of life, and greater mortality.

The autonomic nervous system provides constant control over airway smooth muscle, secretory cells, and vasculature, and therefore, some conventional methods have attempted to treat COPD symptoms by treating portions of the autonomic nervous system. Although sympathetic and parasympathetic branches of the autonomic nervous system innervate the airways, the parasympathetic branch dominates, especially with respect to control of airway smooth muscle and secretions. Cholinergic nerve fibers arise in the nucleus ambiguus in the brain stem and travel down the vagus nerve (right and left vagus nerves) and synapse in parasympathetic ganglia, which are located within the airway wall. These parasympathetic ganglia are most numerous in the trachea and mainstem bronchi, especially near the hilus and points of bifurcations, with fewer ganglia dispersed in distal airways. From these ganglia, short post-ganglionic fibers travel to airway smooth muscle and submucosal glands. Acetylcholine (ACh), the parasympathetic neurotransmitter, is released from post-ganglionic fibers and acts upon M1- and M3-receptors on smooth muscles and submucosal glands to cause bronchoconstriction and mucus secretion, respectively. Acetylcholine may additionally regulate airway inflammation and airway remodeling, and it may contribute significantly to the pathophysiology of obstructive airway diseases.

Wide varieties of stimuli (e.g., cigarette smoke, mechanical stimuli, and other irritants) are able to elicit reflex cholinergic bronchoconstriction through activation of sensory receptors in the larynx or airways. These sensory receptors primarily include rapidly adapting receptors (RARs) and C-Fibers, both of which have nerve endings in the epithelium. Activation of these afferent nerves causes a cholinergic reflex that is known to result in bronchoconstriction and an increase in airway mucus secretion through the activation of muscarinic receptors on airway smooth muscle cells and submucosal glands. Some conventional methods include avoidance of such stimuli and irritants. However, these methods are restrictive and patient compliance is difficult to maintain.

Most conventional treatment methods occur after a patient has been hospitalized following an AECOPD event. This leads to increased trauma to the patient and increased healthcare costs. Therefore, a need exists for methods and systems, which reduce the probability of events such as AECOPD events, by monitoring, predicting, and alerting patients of the onset of AECOPD so that prompt medical attention can be obtained and hospitalization can be avoided. In addition, a need exists for methods and systems, which monitor events associated with COPD (e.g. asthma, cystic fibrosis exacerbations, heart failure exacerbations where the lung may start to fill with fluid (oedema), etc.)

### SUMMARY OF THE DISCLOSURE

In one aspect, the present disclosure is directed to a method of monitoring a lung condition of a patient. The method may include may include automatically receiving a diagnostic signal from one or more sensors delivered adjacent to or within a lung of the patient. The one or more sensors may be active to conduct diagnostic monitoring of the lung. The method also may include calculating a diagnostic value based on the diagnostic signal, and determining, based on the diagnostic value, a change in the lung condition.

Various embodiments of the disclosure may include one or more of the following aspects: the step of determining may include determining, based on the diagnostic value, a change in the lung condition requiring therapy, sending an alert to at least one of the patient and a medical professional, wherein the diagnostic value is permittivity; the one or more sensors may include one or both of: (1) a plurality of sensors external to the lung; and (2) a plurality of sensors implanted in the lung, the one or more sensors may include a plurality of sensors, and may further include a step of measuring the distance between the plurality of sensors; the method may further include a step of determining a patient breathing rate based on a change in the distance between the plurality of sensors; the method may further include a step of comparing the diagnostic value to the patient's previous diagnostic values to determine progression of the lung condition over time; the one or more sensors delivered to the portion of the lungs of the patient may be coupled to an implantable device, the implantable device may be selected from the group consisting of: stents, filters, valves, and catheters; the one or more sensors may be remotely activated; the diagnostic signal from the one or more sensors may be received by an electronic device; the method may further include a step of sending the diagnostic signals over a network for processing prior to the step of calculating the diagnostic value; the method may further include a step of receiving a medical record of the patient over the network in response to sending the diagnostic signal and comparing the diagnostic value to the medical record of the patient; the method may further include a step of determining a location of the patient; the lung condition may be COPD, asthma, cystic fibrosis, heart failure, or sleep apnea.

In another aspect, the present disclosure is directed to a method of monitoring a lung condition of a patient, the method may include steps of scanning one or more portions of a lung of the patient with a scanner, the scanner may include a permittivity sensing portion and a transmitting portion, receiving an electronic signal via the permittivity sensing portion, calculating a permittivity value based on the electronic signal, and determining, based on the permittivity value, a change in the lung condition.

Various embodiments of the disclosure may include one or more of the following aspects: wherein the scanner is an electronic mobile device; the method may further include a step of sending the electronic signal received by the scanner via a network for processing prior to the step of calculating the permittivity value; the method may further include a step of receiving a medical record of the patient over the network in response to sending the electronic signal and comparing the permittivity value to the medical record of the patient; and the method may further include sending an alert via the transmitting portion to the at least one of the patient and a medical professional.

In another aspect, the present disclosure is directed to a system for monitoring a lung condition of a patient, the system may include one or more implantable sensors having a sensing portion configured to sense a parameter indicative of a lung condition and a transmitting portion configured to automatically transmit electronic signals based on the parameter, one or more activators may be configured to remotely activate the one or more sensors and may include a receiver configured to automatically receive the electronic signals from the one or more sensors, and a processor configured to calculate a parameter value based on the electronic signals and determine, based on the parameter value of distance, a change in a lung condition requiring therapy.

Various embodiments of the disclosure may include one or more of the following aspects: the parameter may be permittivity.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 is a schematic view of an exemplary system for monitoring body conditions in accordance with an embodiment of the present disclosure.

FIGS. 2A-2H are side view illustrations of sensor devices in accordance with embodiments of the present disclosure.

FIG. 3 is a schematic illustration of placement of multiple sensors in the lungs of a patient device in accordance with an embodiment of the present disclosure.

FIG. 4 is a flow diagram of a method for monitoring body conditions in accordance with an embodiment of the present disclosure.

### DETAILED DESCRIPTION

Reference will now be made in detail to embodiments of the present disclosure, examples of which are illustrated in the accompanying drawings. Wherever possible, the same reference numbers will be used throughout the drawings to refer to the same or like parts. The present disclosure is directed to a system and method for monitoring body conditions and providing alerts if it is determined a change in body conditions requires therapy.

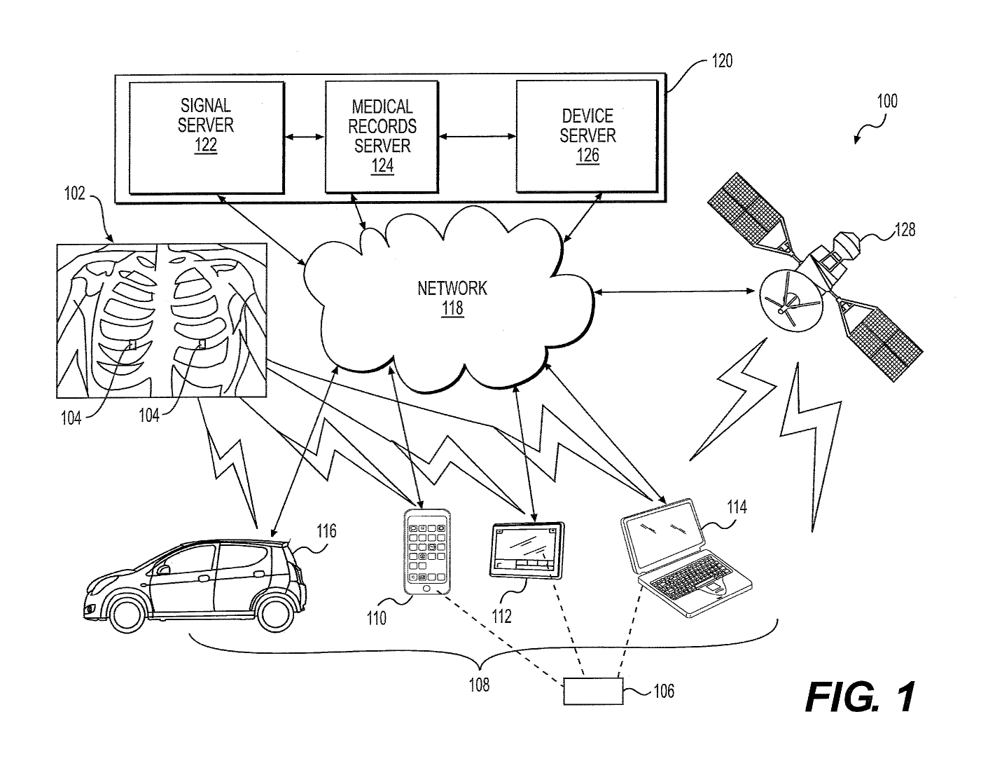

A schematic view of an exemplary system 100 for monitoring lung conditions is illustrated in FIG. 1. The following is a description of the interaction between the components of the system 100 to monitor body conditions and is followed by a further description of each component of the system.

The system 100 may include one or more sensors 104 positioned to receive diagnostic information from a portion of the body 102. The sensors 104 may be positioned on or near a particular portion of the body 102 to be monitored. For example, sensors 104 may be positioned in portions of the lung, heart, throat, or any other suitable location. As will be described in further detail below, the sensors 104 may monitor conditions of a patient's body 102 by performing diagnostic testing at a body location. The sensors 104 may then transmit signals, which may include the diagnostic test data. The signals transmitted by the sensors 104 may be received and processed by a transceiver 106. The transceiver 106 may be located any suitable distance from the sensors 104 and may be internal or external to the body 102. For example, the transceiver 106 may be a component of an electronic device 108 such as a mobile electronic device 110 (e.g. smart phone, watch, etc.), GPS navigation system 112, computer 114, vehicle computers 116, etc. Alternatively, the transceiver may part of a separate dedicated transceiver device e.g. a wand. The transceiver 106 may include a plurality of transceivers 106 such as a network of transceivers 106. The transceiver 106 may process the signals transmitted by the sensors 104 to determine if the diagnostic test data indicates that the patient requires treatment. In some examples, one or more transceivers 106 may be positioned around a building, such as, e.g., a house, hospital, school, to record activations over time. The transceivers 106 may be placed in various implements, accessories, or appliances, such as, e.g., a bed, a door, a chair, or other suitable location. In another example, a mobile electronic device with GPS navigation may be used to simultaneously track the location of a patient, and the activity of the patient via activation of sensor 104.

In some embodiments, the transceiver 106 may only receive or partially process the signals. The partially processed signals may then be sent by the transceiver 106 for further processing by one or more servers, such as a system of servers 120. The transceiver 106 may send the signals to the servers via a network 118, such as the Internet.

The system of servers 120 may include a signal server 122 configured to evaluate the signal, such as the diagnostic test data. The system of servers 120 also may include a medical records server 124 configured to compare the diagnostic test data to the patient's medical records stored in memory, and/or a device server 126 configured to detect the status of the sensors 104. In addition, the system 100 may include a communications satellite 128 configured to transmit data, between various components of the system 100.

The following further describes the various components of the system 100.

As described above, the sensors 104 may detect body conditions and send signals for further processing. The sensors 104 may be any device, which may detect body conditions, and communicate with components external to the body 102.

The sensors 104 may include a detection component configured to detect and measure body conditions, a transmitter configured to communicate signals and/or receive instructions, and a power source. The detection component may perform a diagnostic test by detecting and measuring a property of the body 102. For example, the sensors 104 may detect permittivity, air or blood flow, temperature, breathing rate, impedance, pH, pressure, etc. The sensors may measure body properties in any suitable manner. For example, the sensors 104 may measure permittivity to determine if a lung airway or lung parenchyma is obstructed. Permittivity may be measured by generating an electromagnetic field at a certain radio frequency (RF) and measuring any changes in the RF. In some embodiments, a RF surrogate such as power transmitted to the generated electromagnetic field may be measured and used to determine permittivity. In some embodiments, various frequencies may be used (e.g. UHF range). Frequency Sweep measurements also may be conducted to detect signals.

An increase in permittivity may indicate that the airway is obstructed, and therefore predict an AECOPD event. For example, an airway that is obstructed by tissue and/or fluid may have an increased relative permittivity value (e.g. about 80) compared with an airway with no obstruction e.g. a permittivity value of one (close to vacuum). In another embodiment, the distance between two sensors 104 or between a plurality of sensors implanted in the lungs may be measured to detect breathing rate based on the relative movement of the sensors 104 during inhalation and exhalation, which may be correlated with contraction and expansion of the lungs and also increase/decrease of air in the lungs that may alter permittivity.

The detection component of the sensors 104 may use any suitable technology to perform diagnostic testing such as radio frequency-identification (RFID), temperature probes, optical detectors, Doppler ultrasound, GPS, accelerometers, magnetic sensors (e.g. Hall effect sensors) impedance, electrical, pH, biochemical, pressure, strain, gas (e.g. CO2, O2), air flow, blood flow etc. The sensors 104 also may include a processor, such as a microchip or microcontroller for automatically detecting and processing the detected properties. The processor may be coupled to or be in communication with a memory component for storing diagnostic data and/or other sensor information. The sensors 104 may be activated prior to being positioned at the desired body locations and may begin diagnostic testing immediately upon being positioned at the desired body locations. In other embodiments, the sensors 104 may be programmed to begin diagnostic testing at a pre-determined time. In other embodiments, the sensors 104 may include a receiver that may receive an external activation signal to begin diagnostic testing. Examples of external activation may include an electromagnetic signal, heat activation, or any other form of external activation configured to activate the sensors 104, such as vibration, sonic, ultrasonic, body motion, organ motion, blood motion, air movement, optical, magnetic, electric field, and/or biochemical activation. Sensors 104 also may be configured to track drug compliance and delivery by, e.g., measuring bioavailability among other parameters. In one example, sensors 104 may measure levels of a compound in the tissue by, e.g., optical means, absorption spectra, or the like.

The sensors 104 may be programmed to perform diagnostic testing at a uniform or non-uniform time period. In some embodiments, the sensors 104 may start and stop diagnostic testing in response to an external activator or controller. In some embodiments, periodic diagnostic testing by the sensors may be based on various parameters, such as the health condition of the patient, the time of day, etc. In some embodiments, different sensors 104 at the same location may be programmed to perform different diagnostic testing at the same or different time intervals. The diagnostic testing may occur in accordance with the patient's daily routine. For example, the sensors 104 and/or transceivers 106 may be placed at or near doorways in the patient's home or work, in the patient's car, bed, or any other suitable location so as to gather data as the patient passes by. In this way, additional patient activity data may be gathered to ascertain the general wellbeing of the patient.

In some embodiments, each sensor 104 or set of sensors 104 in the patient may include a unique identifier. Multiple patients can then be tracked by the identifier, such as in a hospital or nursing home etc.

The sensors 104 also may include a communication component configured to transmit and receive signals, such as diagnostic test results, with an external receiver. The communication component of the sensors 104 may include any suitable components such as transmitters, antennas, optical components (e.g. photodiodes, LEDs, lasers, ultrasonic, sonic components), etc. The communication component of the sensors 104 may be coupled with the processor of the sensor 104. The communication component may receive activation signals from an external activator instructing the processor to begin diagnostic testing. In some embodiments, the communication component may receive diagnostic monitoring instructions from an external component. For example, the communication component may receive instructions from an external server, such as the signal server 122 to increase or decrease the frequency of diagnostic testing. The communication component also may send signals saved in the memory component of the sensor to external receivers. In order to conserve power, the communication component may periodically send and receive signals. In other embodiments, the communication component of the sensor 104 may be in constant communication with external receivers. Alternatively, the communication component may only send signals when it receives an activating signal, such an electromagnetic signal from an external receiver. In some embodiments, signals may be exchanged between multiple sensors 104 in the same or different parts of the body 102. Some of the sensors 104 may only send signals, such as diagnostic test data, while other sensors 104 may send and receive signals. In some embodiments, a sensor 104 may receive signals from a plurality of other sensors 104, process the signals, and send a single signal to an external receiver.

The sensors 104 may receive power from any suitable power source. Examples of suitable power sources include internal batteries, such as rechargeable batteries, capacitors, piezoelectric batteries, etc. In addition or alternatively, the power source may include external power sources such as thermoelectric, ultrasonic, optical, or any other suitable external power sources. The external power source may provide power to the sensors 104 each time a diagnostic test is to be performed or the power received from the external source may be stored by the sensor 104 for later use.

The sensors 104 may have any suitable size, shape, and geometry and may have any suitable properties, such as flexibility, radiopacity, sterilizability, biocompatibility, therapeutic (e.g. drug eluting, tissue ablating, etc.) The sensors 104 may be manufactured in any suitable manner using any suitable material or combination of materials. For example, metals or polymers (e.g. polyurethane, polyethylene, PET, PTFE, etc.)

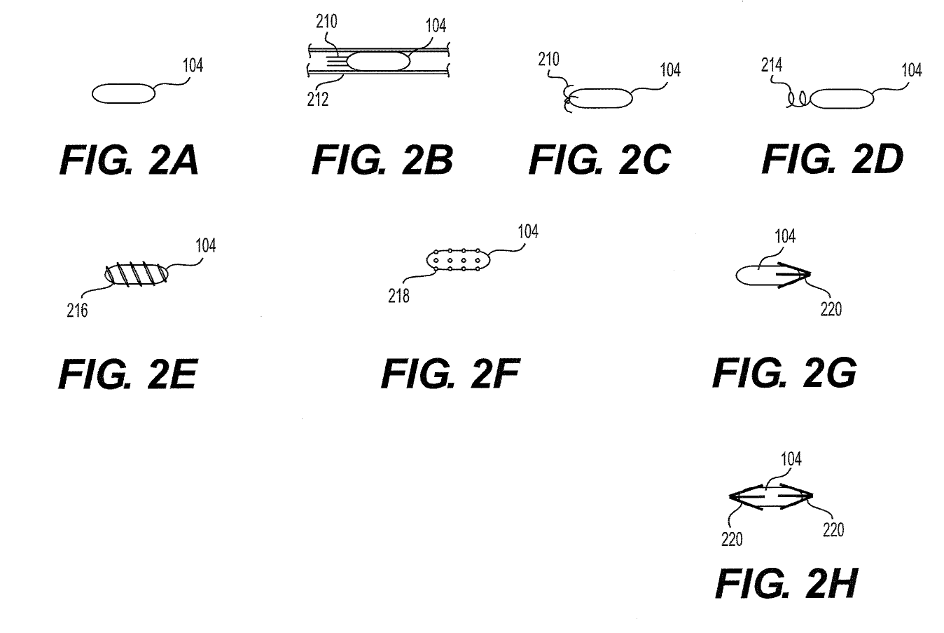

As shown in FIG. 2A, the sensor 104 may be hermetically-sealed within a shell. The hermetically sealed sensors 104 may be thin enough to be inserted into the airway wall bronchoscopically with minimal obstruction to the airway.

FIGS. 2B-2H show embodiments of sensors having various fixation components 210, 214, 216, 218, and 220 configured to prevent the sensor 104 from migrating from a desired location. FIG. 2B shows a sensor 104 placed in an airway 212 and including a fixation component 210 on a portion of the sensor 104. The fixation component 210 may have any suitable shape, size, or geometry to prevent the sensor 104 from migrating from a desired location. For example, as shown in FIGS. 2B and 2C, the fixation component 210 may be formed from a straight shape into a hook shaped barb for anchoring the sensor 104 upon positioning at the desired location. In another embodiment, as shown in FIG. 2D, the fixation component 214 may be on an end portion of the sensor 104 and have a spiral or coil shape. In another embodiment, as shown in FIG. 2E, the fixation component 216 may include threads or raised portions on the outer surface of the sensor which may screw into tissue and retain the sensor 104 at a desired location. In another embodiment as shown in FIG. 2F, the fixation component 218 may include bumps or protrusions on a portion or the entire surface of the sensor 104. In another embodiment, as shown in FIG. 2G, the fixation component may include a collapsible tine on one end of the sensor 104. In another embodiment, as shown in FIG. 2H, the fixation component may include collapsible tines on each end of the sensor 104.

The sensors 104 may be implanted in or on the body 102. For example, multiple sensors 104 may be placed in and/or on the same organ or different organs.

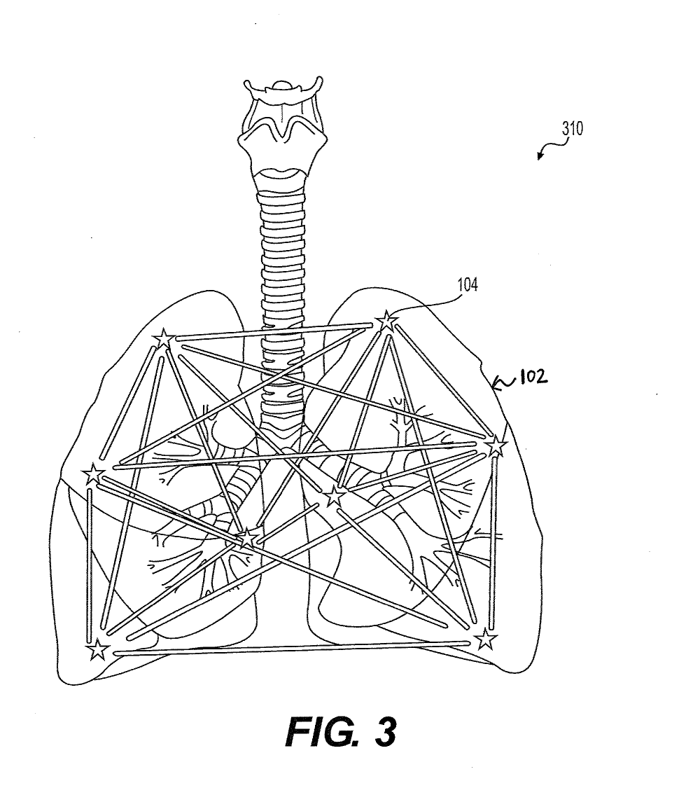

In one embodiment, as shown in FIG. 3, a network 310 of multiple sensors 104 may be placed in different portions of the body 102, such as around or in the lung lobes. Each sensor 104 in the network may detect each other and communicate signals to each other. In this manner, multiple sensors 104 may independently measure the status of different lung lobes. In some embodiments, interpolation of the signal may be performed between the sensors 104 to generate a 3-D map of the lungs for the signal and map the signal over time. In other embodiments, sensors may be placed in the lung lobes and on the skin. Additionally or alternatively, the sensors 104 may be fixed in specific locations within the pleural space.

The sensors 104 may be delivered into the body 102 via any suitable medical delivery devices, such as catheters, scopes, injection, etc. In some embodiments, the sensors may be attached to the skin in any suitable manner, such as via adhesive, sutures, etc. The sensors 104 may be placed directly in or on the body 102. Alternatively, the sensors 104 may be housed in other medical devices. Examples of such other medical devices may include stents, filters, valves, catheters, occlusion devices, drug pumps, electrical stimulators, defibrillators, etc.

As described above, the transceiver 106 may be components of an electronic device such as mobile electronic devices 110, GPS navigation systems 112, computers 114, vehicles 116, etc. The transceiver 106 may include an activating component that may send an activating signal to the sensors 104 in any suitable manner, such as electrical signals, RF, etc. to instruct the sensor 104 to begin diagnostic monitoring. The transceiver 106 may activate the sensor 104 at any suitable distance from the sensors 104. In some embodiments, the transceiver 106 may send an activating signal to sensors 104 having an RFID tag. The RFID tag of the sensors 104 may be activated at a certain predetermined threshold power level that indicates a permittivity level and the transceiver 106 may measure the activation from each sensor 104 and calculate an average or other calculation (e.g. interpolation), and other data that indicates permittivity of a portion of the body 102.

The transceiver 106 also may include a receiving component configured to receive signals detected by/sent by the sensors 104. The receiving component of the transceiver 106 may have any suitable configuration based on the type of signal transmitted by the sensor 104. For example, the receiving component of the transceiver 106 may include a RFID receiver, BLUETOOTH receiver, near field communication receiver, etc.

The transceiver 106 also may include a processor for processing the signals received from the sensors 104. The transceiver processor may partially process or fully process the signals received from the sensors 104. In one embodiment, the transceiver 106 may receive permittivity data from the sensors 104 and process the signals to determine that an airway may be obstructed. In this embodiment, the transceiver 106 may be in a mobile phone 110 or any other electronic device 108. The electronic device 108 may include program instructions, such as a software application, for determining if the signals detected and measured by the sensor 104 are indicative of an event requiring therapy or medical treatment. The electronic device 108 also may include a GPS or other location-detecting component for determining the location of the patient and sending an alert with the location of the patient over the network. The alert may be in any suitable form, such as a SMS message, phone call, and/or email message. The alert may be sent to one or more entities, which may include the patient, emergency services, etc.

In another embodiment, sensors 104 may be placed in an external sensor device, which may be used to scan a patient to perform diagnostic testing and processing of the diagnostic test data. The external device may be any suitable device, such as a wand. In another embodiment, the external sensor device and transceiver 106 may be placed on opposite sides of a patient test site, such as the patient chest. The external sensors may perform external diagnostic testing of a portion of the body 102, such as the lungs, and send signals to the transceiver 106, which may process that signals.

In other examples, the transceiver 106 may receive permittivity signals from the sensors 104, determine that the permittivity values are above a certain pre-determined threshold, and send the processed signals via a network to one or more external servers, such as the network of servers 120. For example, the pre-determined threshold may be established by a percent change over a baseline value. For example, the medical records server 124 may process the signal to determine if the permittivity value is indicative of a medical event (such as AECOPD) requiring intervention. In the above-described examples, the transceiver 106 may include an activation component and a receiver component, however, in other examples, the activator and receiver components may be in separate devices.

The system of servers 120 may be configured to receive signals from the transceiver 106 via the network 118, such as the Internet. The signals may be diagnostic test data detected by the sensors 104 from the body 102. The system of servers may include one or more servers, such as the signal server 122, the medical server 124, and/or the device server 124. In addition, the system of servers 120 may include or be in communication with other servers. The system of servers 120 may by located in the same location, such as a medical care facility, or at different locations. The signal server 122 may be configured to process the signals received from the sensors 104 via the transceiver 106 to determine a diagnostic test value. For example, the signal server 122 may execute one or more programs having algorithms or other calculations for determining a value for the received diagnostic data. In one embodiment, the sensors 104 may detect a permittivity value in the lungs and the signal servers 122 may process the permittivity value to determine if the permittivity value is above a predetermined value that indicates an obstruction in the lungs. Based on the determination, the signal server 122 may send instructions to provide an alert. The medical records server 124 may include a database of the patient's previous diagnostic test results and may include a database of other patient diagnostic test results. The medical records servers 124 may compare the diagnostic test data received from the sensors 104 via the transceiver 106 to the medical records database to determine if the detected diagnostic data is indicative of a medical event (such as AECOPD) requiring treatment. In addition, the medical records server 124 may track the progression of a disease in a patient or cohort of patients over time by comparing the diagnostic test data to previous data. For example, the medical records server 124 may track the extent of emphysema, amount of hyperinflation, and chest remodeling in a patient over time or at an instant. Device servers 126 may detect the performance of the sensors 104 and transceivers 104 and provide updates, maintenance alerts, etc. to maintain the performance of the sensors 104 and transceivers 106.

The sensors 104, transceiver 106, and servers 122, 124, and 126 may include a data communication interface for packet data communication and a central processing unit (CPU), in the form of one or more processors, for executing program instructions, such as programs for analyzing permittivity data. These components also may include an internal communication bus, program storage, and data storage for various data files to be processed and/or communicated by the transceiver 106 such as ROM and RAM, although sensors 104, transceiver 106, and servers 122, 124, and 126, also may receive programming and data via network communications. The hardware elements, operating systems, and programming languages of sensors 104, transceiver 106, and servers 122, 124, and 126 may be conventional in nature, and it is presumed that those skilled in the art are adequately familiar therewith. The transceiver 106 and servers 122, 124, and 126 also may include input and output ports to connect with input and output devices such as keyboards, mice, touchscreens, monitors, displays, etc. Of course, the various functions may be implemented in a distributed fashion on a number of similar platforms, to distribute the processing load.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of a communication network into the computer platform of a server and/or from a server to the transceiver 106. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

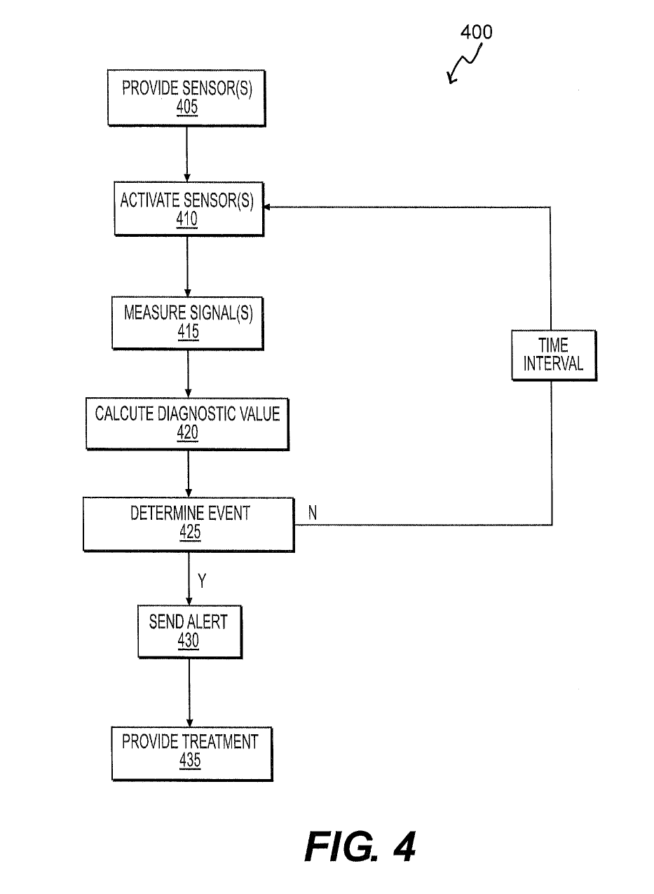

FIG. 4 shows a flow diagram of a method 400 for monitoring body conditions in accordance with an embodiment of the present disclosure. The method may be used for detecting and/or predicting events requiring medical treatment, such as AECOPD.

The method and system described above also may be used to detect and/or predict other medical conditions and/or events, such as heart failure exacerbations where the lung may start to fill with fluid (oedema), fluid accumulation in the limbs and abdomen, and any other event and/or conditions.

As shown in FIG. 4, the method 400 may include a step 405 of providing one or more sensors at a portion of the patient's body 102. The sensors 104 may be implanted in any portion or portions of the body 102 (such as proximate airways of the lung) and/or placed on the body 102 in any suitable manner as discussed above. For example, the sensors 104 may be positioned anywhere surrounding a lung or a target portion of the lung, e.g. within the inner lumen of the bronchi, in the wall of the bronchi, in the parenchyma, in a bulla, in the pleural space, or in other locations outside the lung and within the thoracic cavity. In some embodiments, the sensors 104 may be positioned in the trachea for performing cough measurement. In some embodiments, the sensors 104 may be positioned anywhere in the lung. For example, for a COPD patient, it may be desirous to ‘capture’ the whole lung, unless there are particular lobes that are more problematic for that patient (which may be determined by the healthcare provider via CT scan, etc.), which may then be tracked.

At step 410, the sensors 104 may be activated by an activator such as a transceiver 106 in any suitable manner as discussed above. The sensors 104 may be activated a single time to begin diagnostic monitoring. In other embodiments, the sensors 104 may be activated each time a diagnostic test is to be performed or periodically (e.g. each day, each week, each month). In some embodiments, the sensors 104 may continuously record information and then retrieve (e.g. download) the stored date at the activation times.

A signal may be measured by the sensors 104 at step 415. The signal may include any diagnostic test or combination of diagnostic tests, such as permittivity, temperature, impedance, pressure, pH, distance between sensors, etc. The signal may be transmitted to one or more external receivers, such as the transceivers 106. The transceivers 106 may be components in electronic devices 110 or may be placed in various locations around the patient's house, workplace, car 116, etc. The signal may be measured each time the user passes by a transceiver 106. For example, one sensor 104 may measure breathing rate by complex permittivity measurement through the lung, as the permittivity changes may be proportional to the inverse of lung volume.

In some embodiments, an algorithm may use multiple parameters to determine a medical event, e.g. an algorithm may be used to combine various parameters (e.g. activity level+respiration rate+distance between sensors+permittivity) to determine onset of AECOPD. The distance between sensors and activity level may differentiate changes in permittivity due to hyperinflation/air trapping in the lungs vs. increased tidal volume realized during exercise.

Based on the signal, a diagnostic value may be calculated at step 420. The diagnostic value may be calculated by the sensors 104, the transceiver 106, and/or the system of servers 120. The diagnostic value may be calculated based on any algorithm or calculation stored in memory and automatically electronically executed by a processor. At step 425, it may be determined if the diagnostic property calculated at step 425 is indicative of a medical event.

Step 425 may include comparing the diagnostic value to a threshold value, past medical events of the patient or other patients, or any other data saved in memory of the sensors 104, transceiver 106, and/or system of servers 120. For example, the diagnostic value may be compared to a baseline value for the patient. The baseline value may be generated for a particular patient over a period of time (e.g. hours, days, weeks) when it is known that the patient is in a stable condition. Any changes relative to the baseline value (e.g. above or below the baseline value) within a pre-determined standard deviation that is maintained over a pre-determined amount of time (e.g. hours, 1 hour, 30 minutes, 5 minutes, 30 seconds, etc.) may be indicative of an event. Calibration of the sensors 104 for each patient may be performed by taking measurements at maximum inspiration and maximum exhalation when the patient is healthy or after inhaling a bronchodilator. Changes after such measurements such as due to fluid buildup/oedema may shift the measured baseline.

If it is determined that the diagnostic property calculated at step 425 is indicative of a medical event, then an alert may be sent at step 430 to the patient, physicians, and/or emergency services, and treatment may be provided at step 435. If it is determined that the diagnostic property calculated at step 425 is not indicative of a medical event, then steps 410-425 may be repeated after any suitable predetermined time interval. In some embodiments, the alert may be send to a handheld device (e.g. smart phone) which may send the patient a query to determine the medical state of the patient and/or the deviation from the baseline value is due to a non-medical event (e.g. exercise, startle, etc.). For example, the patient may be queried as to whether if the patient is having any other symptoms (e.g. shortness of breath) so as to further assess if the triggered alert is indeed an event requiring medical care.

In some embodiments, the sensors 104 may be used to monitor atrial fibrillation, heartbeat, ECG, etc. In some embodiments, the sensors 104 may be used to monitor the progression of disease. For example, the sensors 104 may be used to monitor the progression of lung cancer, and/or monitor the status of patients who may be at risk for lung cancer so as to provide an early alert and treatment. In this example, as tumors develop, the tumors may create a large change in permittivity that may send an alert to have the patient checked by a healthcare provider.

Any aspect set forth in any embodiment may be used with any other embodiment set forth herein. The devices and apparatus set forth herein may be used to monitor any suitable condition at any suitable portion of the body.

It will be apparent to those skilled in the art that various modifications and variations can be made in the disclosed systems and processes without departing from the scope of the invention. Other embodiments of the invention will be apparent to those skilled in the art from consideration of the specification and practice of the invention disclosed herein. It is intended that the specification and examples be considered as exemplary only.

We claim:

1. A method of monitoring a lung condition of a patient, the method comprising:
automatically receiving a diagnostic signal from one or more sensors delivered adjacent to or within a lung of the patient, the one or more sensors being active to conduct diagnostic monitoring of the lung,
calculating a diagnostic value based on the diagnostic signal, and
determining, based on the diagnostic value, a change in the lung condition.

2. The method of claim 1, wherein the step of determining comprises:
determining, based on the diagnostic value, a change in the lung condition requiring therapy, and
sending an alert to at least one of the patient and a medical professional.

3. The method of claim 1, wherein the diagnostic value is permittivity.

4. The method of claim 1, wherein the one or more sensors comprises one of: (1) a plurality of sensors external to the lung; and (2) a plurality of sensors implanted in the lung.

5. The method of claim 1, wherein the one or more sensors comprise a plurality of sensors, and further comprising a step of measuring the distance between the plurality of sensors.

6. The method of claim 5, further comprising a step of determining a patient breathing rate based on a change in the distance between the plurality of sensors.

7. The method of claim 1, further comprising a step of comparing the diagnostic value to the patient's previous diagnostic values to determine progression of the lung condition over time.

8. The method of claim 1, wherein the one or more sensors are remotely activated.

9. The method of claim 1, wherein the one or more sensors delivered to the lung of the patient are coupled to an implantable device, wherein the implantable device is selected from the group consisting of: stents, filters, valves, electrical stimulation devices, pumps, and catheters.

10. The method of claim 1, wherein the diagnostic signal from the one or more sensors is received by an electronic device.

11. The method of claim 1, further comprising a step of sending the diagnostic signals over a network for processing prior to the step of calculating the diagnostic value.

12. The method of claim 11, further comprising a step of receiving a medical record of the patient over the network in response to sending the diagnostic signal and comparing the diagnostic value to the medical record of the patient.

13. The method of claim 1, further comprising a step of determining a location of the patient.

14. The method of claim 1, wherein the lung condition is selected from the group consisting of: COPD, asthma, cystic fibrosis, heart failure, and sleep apnea.

15. A method of monitoring a lung condition of a patient, the method comprising:
scanning one or more portions of a lung of the patient with a scanner, the scanner comprising a permittivity sensing portion and a transmitting portion,
receiving an electronic signal via the permittivity sensing portion,
calculating a permittivity value based on the electronic signal, and
determining, based on the permittivity value, a change in the lung condition.

16. The method of claim 15, wherein the scanner is an electronic mobile device.

17. The method of claim 15, further comprising a step of sending the electronic signal received by the scanner via a network for processing prior to the step of calculating the permittivity value.

18. The method of claim 17, further comprising a step of receiving a medical record of the patient over the network in response to sending the electronic signal and comparing the permittivity value to the medical record of the patient.

19. The method of claim 15, further comprising sending an alert via the transmitting portion to the at least one of the patient and a medical professional.

20. A system for monitoring a lung condition of a patient, the system comprising:
one or more implantable sensors having a sensing portion configured to sense a parameter indicative of a lung condition and a transmitting portion configured to automatically transmit electronic signals based on the parameter,
one or more activators configured to remotely activate the one or more sensors and comprising a receiver configured to automatically receive the electronic signals from the one or more sensors, and
a processor configured to calculate a parameter value based on the electronic signals and determine, based on the parameter value, a change in a lung condition.
